(19) United States
(12) Patent Application Publication
(10) Pub. No.: US 2021/0228766 A1
(43) Pub. Date: Jul. 29, 2021

(54) MICROSPHERE FOR EMBOLIZATION, PREPARATION METHOD THEREOF, AND METHOD FOR EMBOLIZING TUMOR USING THE SAME

(71) Applicant: T-ACE Medical Co., Ltd., Tainan (TW)

(72) Inventors: CHIH HONG CHEN, Tainan (TW); CHAU NAN HONG, Tainan (TW); CYUN JHE YAN, Tainan (TW); CHUEH KUAN WANG, Tainan (TW); CHEN HSI CHOU, Tainan (TW); YI SHENG LIU, Tainan (TW); HONG MING TSAI, Tainan (TW); CHI MING HO, Tainan (TW); CHUAN SHENG LIN, Tainan (TW); XI ZHANG LIN, Tainan (TW)

(73) Assignees: T-ACE Medical Co., Ltd., Tainan (TW); T-ACE Medical Co., Ltd., Tainan (TW)

(21) Appl. No.: 17/051,767

(22) PCT Filed: May 31, 2018
(86) PCT No.: PCT/CN2018/089270

§ 371 Date: Oct. 29, 2020

(51) Int. Cl. A61L 24/04; A61L 24/00

(52) U.S. Cl. CPC A61L 24/043; A61L 24/0015; A61L 2300/404; A61L 2300/802; A61L 2300/622; A61L 2300/216; A61L 2300/21; A61L 2400/04; A61L 2300/10

(57) ABSTRACT

A microsphere includes a cross-linked hydrophilic substrate, a lipophilic substrate, and a surfactant. The cross-linked hydrophilic substrate includes cross-linked sodium alginate and gelatin. The lipophilic substrate includes iodized oil, C16-C18 alkyl alcohol, and polycaprolactone. The surfactant includes polyoxyethylene stearate. The microsphere is dry or substantially solid. Prior to being used for embolization, the microsphere can be immersed in a drug containing mixture to allow the microsphere to absorb the mixture and expand, thereby loaded with the drug. A method for preparing the microsphere and a method for embolizing tumor in a subject by using the microsphere are also provided.

### BACKGROUND OF THE INVENTION

### Technical Field

The present invention relates to medicinal products, and in particular, to a microsphere with high hydrophilicity for embolizing blood flow in blood vessels, a preparation method thereof, and a method for embolizing tumor by using the microsphere.

### Background

Cancer or tumors are diseases causing extremely high mortality in humans. Nowadays, treatment methods for cancer or tumors include surgical treatment (tumor resection), internal medicinal treatment (transcatheter arterial embolization (TAE), and percutaneous ethanol injection (PEI)), and supporting treatment (cryotherapy, radiotherapy, and chemotherapy). Among the above-mentioned treatment methods, the treatment rationale of TAE involves the fact that since nutrients for cancer or tumor tissues are supplied by the arteries, once the arteries are embolized, the cancer or tumor tissues would necrose due to lack of nutrients. TAE is particularly suitable for vascular tumors or hyperproliferative tumors, such as liver cancer, kidney cancer, spleen enlargement, prostatic hyperplasia or hysteromyoma.

The embolizing compositions currently used in TAE typically include degradable materials (such as gelatin) and non-degradable materials (such as polyvinyl alcohol (PVA), vinyl resin, and drug eluting beads (DEB)). Among, the compositions, gelatin is relatively low in price, but is unable to effectively carry chemotherapeutics, resulting in a poor therapeutic effect. If non-degradable materials are used as the embolizing compositions, chemotherapeutics can be effectively loaded; however, the price is expensive, and the non-degradable materials cannot be degraded in vivo, even causing cancer cells to generate drug resistance or other similar responses, and resulting in a poor therapeutic effect. in addition, none of the above-mentioned embolizing compositions has X-ray contrasting properties, making it impossible to track the location of the embolizing compositions in vivo.

In order to alleviate the above-mentioned problems, Taiwan Patent No. TWI503132 discloses a pharmaceutical microsphere for embolization, which has biodegradable and X-ray contrasting properties and is capable of loading a drug. However, due to the lipophilicity of the pharmaceutical microsphere, in cases where a drug needs to be loaded, the drug must be added in during the preparation process of the pharmaceutical microsphere; it is not possible to freely add any desired drug according to the needs of a back-end user after the pharmaceutical microsphere is produced. Furthermore, as the pharmaceutical microsphere is lipophilic, loading a hydrophilic drug to the pharmaceutical microsphere has not been effective. In view of the aforementioned, for development of a microsphere that allows the back-end user to freely add a desired drug, there is still a need to further improve the pharmaceutical microsphere for embolizing blood flow in blood vessels.

### SUMMARY

The present invention provides a microsphere for embolizing blood flow in blood vessels that enables a back-end user to freely add a desired drug as required, and has both biodegradable and X-ray contrasting properties.

Thus, an embodiment of the present invention provides a dry or substantially solid microsphere. The microsphere includes:

a cross-linked hydrophilic substrate that includes cross-linked sodium alginate and gelatin,

a lipophilic substrate that includes iodized oil, C16-C18 alkyl alcohol, and polycaprolactone, and

a surfactant that includes polyoxyethylene stearate.

Another embodiment of the present invention further provides a method for preparing the microsphere, including:

mixing the cross-linked hydrophilic substrate, the lipophilic substrate and the surfactant to obtain an emulsion; and

granulating the emulsion to obtain the microsphere.

Yet another embodiment of the present invention provides a method for embolizing tumor in a subject by using the microsphere.

### BRIEF DESCRIPTION OF THE DRAWINGS

FIG. 1 are images showing the dissolution of a drug-loaded microsphere according to an embodiment of the present invention;

FIG. 2 are images showing embolization using the drug-loaded microsphere according to an embodiment of the present invention;

FIG. 3 are computed tomography images of a liver on day 0, day 4, day 12, and day 25 after embolization by the drug-loaded microsphere according to an embodiment of the present invention; and

FIG. 4 are exemplary images showing changes of an organ on day 35 after embolization by the drug-loaded microsphere according to an embodiment of the present invention.

### DETAILED DESCRIPTION

The present invention provides a microsphere for embolizing blood flow in blood vessels that allows a back-end user to freely add a desired drug as required, and has both biodegradable and X-ray contrasting properties.

Thus, an embodiment of the present invention provides a dry or substantially solid microsphere. The microsphere includes:

a cross-linked hydrophilic substrate that includes cross-linked sodium alginate and gelatin;

a lipophilic substrate that includes iodized oil, C16-C18 alkyl alcohol, and polycaprolactone, and

a surfactant, including polyoxyethylene stearate.

Another embodiment of the present invention further provides a method for preparing the microsphere, including:

mixing the cross-linked hydrophilic substrate, the lipophilic substrate and the surfactant to obtain an emulsion; and

spray-granulating the emulsion to obtain the microsphere.

The “microsphere” described herein may be of any shape, for example: a spherical shape, a near-spherical shape, a conical shape, a columnar shape, a cubic shape, or an irregular shape; a spherical shape is preferred. On the other hand, the microsphere is substantially solid or colloid, such as dry powdered solid, dry granular solid, or water-containing colloid. In a preferred embodiment of the present invention, the average particle size of the microsphere is 40 to 1000 μm, preferably 100 to 750 μm, more preferably 150 to 350 μm.

The “cross-linked hydrophilic substrate” described herein includes cross-linked sodium alginate and gelatin. Without intending, to be limited by any particular theory, it is believed that the cross-linked hydrophilic substrate gives the microsphere used for embolizing blood flow in blood vessels its high hydrophilicity. In the cross-lined hydrophilic substrate, sodium alginate is the main source of negative charges for the microsphere for embolizing blood flow in blood vessels. The negative charges allow the microsphere for embolizing blood flow in blood vessels to be morphologically stable and bind various ingredients. In addition, sodium alginate has the properties of an anionic emulsifier, which improve the texture of the microsphere for embolizing blood flow in blood vessels, and increase the viscosity and stability of the microsphere for embolizing blood flow in blood vessels. On the other hand, gelatin can increase the viscosity of the microsphere for embolizing blood flow in blood vessels. During the preparation process, part of the gelatin and the supplemental components of the microsphere for embolizing blood flow in blood vessels, such as sucrose, sorbitol and glycerol, would form a capsule-like substance, which blocks the flow of oily compounds by encapsulation.

The cross-linked sodium alginate and gelatin may self-cross-link or cross-link with each other to form a substrate, or may cross-link by using other cross-linking agents to form a substrate. In this case, the cross-linked hydrophilic substrate further includes a cross-linking agent, preferably a calcium ionic crosslinking agent, more preferably calcium chloride or calcium lactate. In a preferred embodiment of the present invention, the dry weight of the calcium ionic cross-linking agent is 0.01 wt %, when the total dry weight of the microsphere is 100 wt %.

In a preferred embodiment of the present invention, the dry weight of the cross-linked hydrophilic substrate is 50 to 70 wt %, preferably 55 to 65 wt %, more preferably 55 to 60 wt %, when the total dry weight of the microsphere is 100 wt %.

Preferably, the dry weight of sodium alginate is 35 to 45 wt %, more preferably 39 wt %, and the dry weight of gelatin is 15 to 25 wt %, more preferably 20 wt %, when the total dry weight of the microsphere is 100 wt %.

The “lipophilic substrate” described herein includes iodized oil, C16-C18 alkyl alcohol, and polycaprolactone. Without intending to be limited by any particular theory, it is believed that in addition to providing lipophilicity, iodized oil may also provide the microsphere for embolizing blood flow in blood vessels an image contrasting effect, as well as a blood flow blocking function that causes necrosis of tumor cells. C16-C18 alkyl alcohol is an emulsifier that facilitates homogenization with the cross-linked hydrophilic substrate. C16-C18 alkyl alcohol may be linear or branched, and is preferably hexadecanol or octadecanol. Polycaprolactone has been used in surgical suture; it is degradation resistant and can prolong the duration of time of the lipophilic substrate in blocking blood flow in blood vessels. In addition, polycaprolactone can also promote binding (e.g., via electrostatic interaction) between the microsphere and a therapeutic agent, thereby improving drug loading rate, controlling and sustaining the release of the therapeutic agent, and preventing the microsphere for embolizing blood flow in blood vessels from disintegration and loss of activity.

In a preferred embodiment of the present invention, the dry weight of the lipophilic substrate is 18 to 30 wt %, preferably 20 to 28 wt %, more preferably 22 to 26 wt %, when the total dry weight of the microsphere is 100 wt %.

Preferably, the dry weight of the iodized oil is 6 to 10 wt %, more preferably 10 wt %; the dry weight of C16-C18 alkyl alcohol is 6 to 12 wt %, more preferably 8 wt %; and the dry weight of the polycaprolactone is 6 to 9 wt %, more preferably 8 wt %; when the total dry weight of the microsphere is 100 wt %.

The “surfactant” described herein includes polyoxyethylene stearate, preferably polyoxyethylene (40) stearate. Without intending to be limited by any particular theory, it is believed that the surfactant is an emulsifier at high temperature (e.g., higher than 47° C.), allowing miscibility of the lipophilic substrate with the hydrophilic substrate; the surfactant is solid at low temperature (e.g., lower than 47° C.), constituting a part of the microsphere,

In a preferred embodiment of the present invention, the dry weight of the surfactant is 4 to 8 wt %, preferably 5 to 7 wt %, more preferably 6 wt %, when the total dry weight of the microsphere is 100 wt %.

In a preferred embodiment of the present invention, the microsphere for embolizing blood flow in blood vessels further includes a supplemental component. Preferably, the supplemental component includes, but is not limited to, a cosolvent, an antioxidant, an antibacterial agent, and a stabilizer.

In a preferred embodiment of the present invention, the dry weight of the supplemental component is 6 to 20 wt %, when the total dry weight of the microsphere is 100 wt %.

The cosolvent can increase the solubility of sodium alginate in the emulsion and the viscosity of the microsphere for embolizing blood flow in blood vessels. Examples of the cosolvent include, but are not limited to, sucrose, sorbitol, or glycerol. In a preferred embodiment of the present invention, the dry weight of the cosolvent is 1 to 15 wt %, preferably 2 to 10 wt %, more preferably 3 to 9 wt %, when the total dry weight of the microsphere is 100 wt %. Preferably, the dry weight of sucrose is 1 to 5 wt %, more preferably 2 wt %; the dry weight of sorbitol is 2 to 5 wt %, more preferably 3 wt %; and the dry weight of glycerol is 2 to 5 wt %, more preferably 4 wt %.

Examples of the antioxidant include, but are not limited to, sodium thiosulfate or 2,6-di-tert-butyl-p-cresol. In a preferred embodiment of the present invention, the dry weight of the antioxidant is 0.02 to 0.2 wt %, preferably 0.02 to 0.1 wt %, when the total dry weight of the microsphere is 100 wt %. Preferably, the dry weight of sodium thiosulfate is 0.09 wt %, and the dry weight of 2,6-di-tert-butyl-p-cresol is 0.03 wt %.

The antibacterial agent can effectively inhibit the growth of bacteria in the emulsion. Examples of the antibacterial agent include, but are not limited to, propyl p-hydroxybenzoate. In a preferred embodiment of the present invention, the dry weight of the antibacterial agent is 0.1 to 0.5 wt %, preferably 0.2 to 0.4 wt %, more preferably 0.2 wt %, when the total dry weight of the microsphere is 100 wt %.

The stabilizer can stabilize the emulsion. Examples of the stabilizer include, but are not limited to, cholesterol or sodium acetate. As a stabilizer, cholesterol enables the cross-linked hydrophilic substrate and the lipophilic substrate to be more stably combined without separation. Sodium acetate can increase negative charges of the microsphere for embolizing blood flow in blood vessels, and stabilize the overall pH of the emulsion. In a preferred embodiment of the present invention, the dry weight of the stabilizer is 0.2 to 4.5 wt %, preferably 0.5 to 3.5 wt %, more preferably 0.7 to 2.5 wt %, when the total dry weight of the microsphere is 100 wt %. Preferably, the dry weight of cholesterol is 0.5 to 3.5 wt %, more preferably 1.5 wt %; and the dry weight of sodium acetate is 0.2 to 1.0 wt %, more preferably 0.5 wt %.

In a preferred embodiment of the present invention, the microsphere for embolizing blood flow in blood vessels further includes a drug-containing mixture, and can function as a drug release system. The drug in the mixture may be hydrophilic or lipophilic, preferably lipophilic, and may bind with the lipophilic substrate of the microsphere. On the other hand, the drug is not limited to any particular drug, and can be any drug known in the art that may be used for the treatment of diseases in patients; the drug is preferably a radioactive compound, a lipid-soluble drug, or a water-soluble drug. Drugs for treating tumors or cancer may include doxorubicin, bevacizumab, sorafenib, resveratrol or curcumin.

Another embodiment of the present invention provides a method for preparing the microsphere for embolizing blood flow in blood vessels, including:

mixing the cross-linked hydrophilic substrate, the lipophilic substrate and the surfactant to obtain an emulsion; and

granulating the emulsion to obtain the microsphere.

The granulation process is not limited to any particular granulation technique; any granulation technique that can produce microspheres, such as spray granulation. can be used.

Preferably, the method further includes a drying step after granulation. In the drying step, water undischarged during the granulation process is removed, so as to facilitate the preservation of the microsphere for embolizing blood flow in blood vessels. In the drying step, such as (but not limited to) in freeze-drying, the weight of the water removed by freeze-drying is about 18 to 19 times the weight of the resulting dry microsphere.

Preferably, the method further includes: immersing the dry microsphere in a drug-containing mixture to allow the dry microsphere to absorb the mixture and expand. Preferably, the drug-containing mixture is a solution for intravascular injection, in which the microsphere for intravascular embolization absorbs the solution and expand and uniformly disperses in the solution for intravascular injection. When in use, the level of expansion of the microsphere for embolizing blood flow in blood vessels can be adjusted as required; and the level of expansion varies by the ion concentration of the solution for intravascular injection. In an embodiment, the weight ratio of the drug (e.g., doxorubicin) to the dry microsphere may be at least 1:2.5.

Yet another embodiment of the present invention provides uses of the microsphere, in which the microsphere is used for preparing a medical product for embolizing blood flow in blood vessels. Without intending to be limited by any particular theory, it is believed that by combining the cross-linked hydrophilic substrate with the other components, the microsphere for embolizing blood flow in blood vessels has high hydrophilicity, and a desired drug can be freely added to the microsphere according to the needs of back-end users. The microsphere for embolizing blood flow in blood vessels achieves its therapeutic purpose by using physically embolizing blood flow in blood vessels.

Preferably, the microsphere is used for preparing a medical product for treating a tumor by embolizing blood flow in blood vessels. More preferably, the tumor is a hypervascular tumor or a hyperproliferative tumor. The hypervascular tumor includes, but is not limited to, liver cancer or kidney cancer. The hyperproliferative tumor includes, but is not limited to, spleen enlargement, prostatic hyperplasia, or hysteromyoma.

Still another embodiment of the present invention provides a method for embolizing tumor in a subject by using the microsphere. The method includes: immersing a dry microsphere in a mixture to allow the dry microsphere to absorb the mixture and expand; and administering a therapeutically effective amount of the microsphere to the tumor. The mixture may include a drug for treating the tumor.

The following non-limitative embodiments will help a person skilled in the art to implement the present invention. The embodiments should not be considered as limiting the present invention. A person skilled in the art may make modifications and variations on the embodiments discussed in this specification without departing from the spirit or scope of the present invention, and such modifications and variations shall fail within the scope of the present invention.

### EMBODIMENTS

Preparation of Microspheres for Embolizing Blood Flow in Blood Vessels

The content of each component of the microsphere is shown in Table 1 below:

TABLE 1

| Components | Dose (g) | Example 1 | Example 2 | Example 3 | Example 4 |
|---|---|---|---|---|---|
| Sodium alginate | 10.8-13.9 | 11.9 | 11.9 | 11.9 | 11.9 |
| Gelatin | 4.6-7.7 | 6.0 | 6.0 | 6.0 | 6.0 |
| Iodized oil | 1.9-3.1 | 2.4 | 3.1 | 2.4 | 2.4 |
| Hexadecanol | 0.9-1.9 | 1.2 | 1.2 | 1.2 | 1.2 |
| Octadecanol | 0.9-1.9 | 1.2 | 1.2 | 1.2 | 1.2 |
| Polycaprolactone | 1.9-2.8 | 2.4 | 2.4 | 2.4 | 2.4 |
| Polyoxyethylene (40) stearate | 1.2-2.5 | 1.8 | 1.8 | 1.8 | 1.8 |
| Sucrose | 0.6-1.5 | 0.25 | 0.45 | 0.65 | 0.45 |
| Sorbitol | 0.6-1.5 | 1.175 | 0.975 | 0.875 | 0.655 |
| Glycerol | 0.6-1.5 | 0.975 | 1.2 | 0.875 | 1.295 |
| 2,6-di-tert-butyl-p-cresol | 0.0-0.1 | 0 | 0.009 | 0 | 0 |
| Propyl p-hydroxybenzoate | 0.0-0.2 | 0 | 0.06 | 0.12 | 0.18 |
| Cholesterol | 0.2-0.9 | 0.45 | 0.45 | 0.35 | 0.30 |
| Sodium acetate | 0.1-0.5 | 0.15 | 0.15 | 0.25 | 0.30 |

Solution A: Sodium alginate, sucrose, sodium acetate, and propyl p-hydroxybenzoate were weighed according to Table 1 above, and were mixed with 440 mL of distilled water. The solution was water-heated at 90 to 100° C., and stirred at the speed of 325 to 350 rpm for 1 to 1.5 h.

Solution B: Iodized oil, cholesterol, hexadecanol, octadecanol, polycaprolactone, polyoxyethylene (40) stearate, and 2,6-di-tert-butyl-p-cresol were weighed according to Table 1 above, and mixed. The solution was water-heated at 90 to 100° C., and stirred at the speed of 200 rpm for 20 to 30 min.

Solution C: Gelatin, glycerol, and sorbitol were weighed according to Table 1 above, and mixed with 60 mL of distilled water. The solution was water-heated at 90 to 100° C., and stirred at the speed of 200 rpm for 20 to 30 min

Microsphere collection solution: 75 g of calcium chloride was added into 10 L of distilled water; the solution was water-heated at 90 to 100° C., and stirred at the speed of 300 to 400 rpm.

Solution C and Solution B were sequentially added to Solution A. The mixture was heated at 90 to 100° C., and stirred at the speed of 325 to 350 rpm for 1 to 135 h.

A continuous injection pump was set at a speed of 18 mL/min and a volume of 8 mL.

A hot air spray granulator was set a hot air flow rate of 2.4 L/min and an internal pressure of 1 psi/kg/cm³.

The injection pump and hot air spray granulator were turned on and sprayed for 20 to 30 min to generate granules.

Sterilized screen meshes of 177, 149, 125, 104, 74 and 63 μm were arranged from top to bottom in the descending order according to the mesh numbers, and are fixed on a vibrating screener. The microspheres sprayed into the calcium chloride collection solution were sucked out by a suction device and placed on the screen meshes for vibration screening. Each of the screen meshes was rinsed with 4000 mL of sterilized water, each was removed after being rinsed, following by rinsing the subsequent screen mesh. When the rinsing was completed, the microspheres were scraped down by a spatula, and freeze-dried for 48 h. During the freeze-drying, vacuum was turned on when the temperature dropped to −40 to −45° C.

The resulting microsphere for embolizing blood flow in blood vessels has an average particle size of 40 μm to 1000 μm.

Preparation of Drug-loaded Microspheres for Embolizing Blood Flow in Blood Vessels

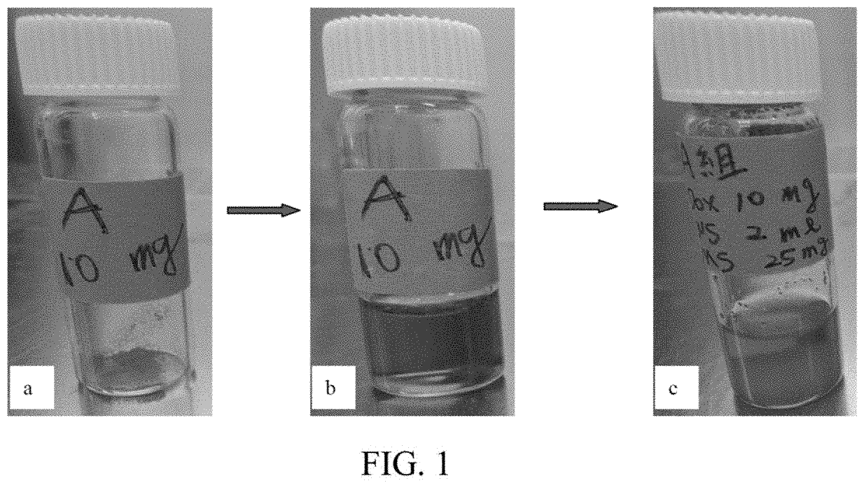

As shown in FIG. 1, 10 mg of doxorubicin was placed in an empty vial (FIG. 1a). Thereafter, 2 ml of normal saline was added. The two uniformly dissolved in about 10 min. Then, the solution is drawn out by using a syringe, and placed into another vial containing 25 mg of microspheres prepared according to Example 2 (FIG. 1b). The 25 mg of microspheres absorbed the mixture of normal saline and doxorubicin for 15 min, precipitated on the bottom of the vial (FIG. 1c).

Embolization of blood flow in blood vessels for treating cancer

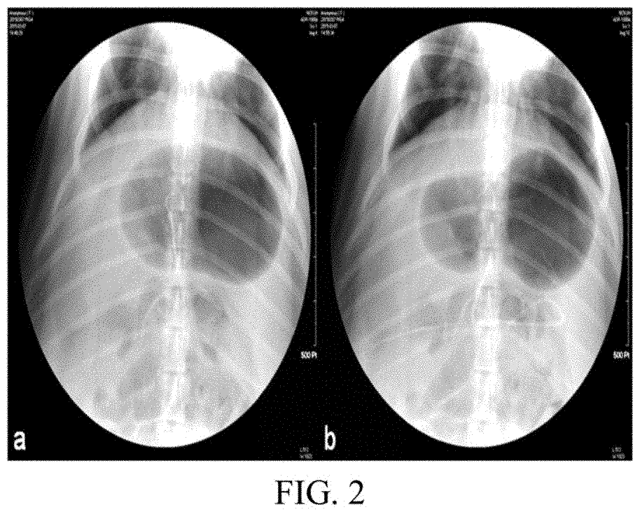

In the example of embolization using the drug-loaded microspheres as shown in FIG. 2, embolization catheters were guided under X-ray to the liver (FIG. 2a) and the spleen (FIG. 2b) of a pig for embolization. At the left lobe of the liver, 0.3 g of hydrophilic microspheres containing, 50 mg/10 ml doxorubicin injection solution was administered for embolization (FIG. 2a), and at the spleen, 0.15 g of the hydrophilic microspheres was administered for embolization (FIG. 2b).

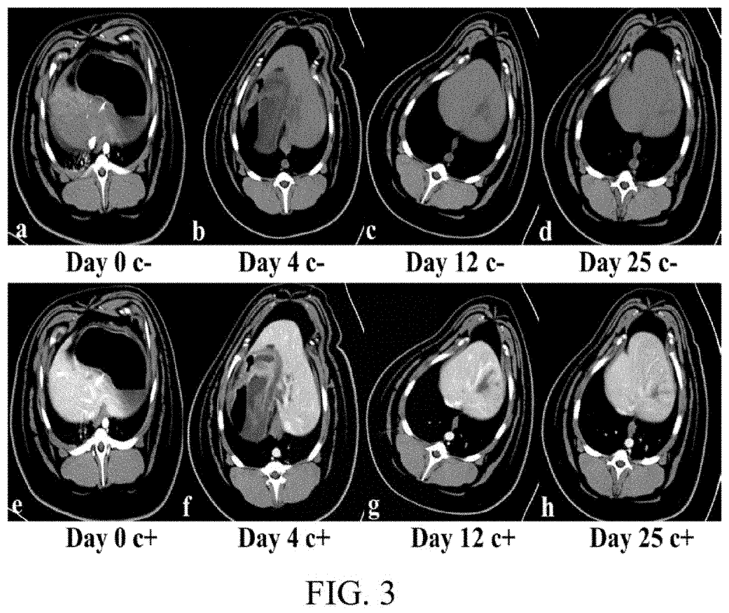

As shown in FIG. 3, after the embolization with drug-loaded microspheres, computed tomography imaging was carried out on the liver of the pig on day 0, day 4, day 12, and day 25. In FIG. 3, “c+” represents injection of a contrasting agent, and “c−” represents the absence of any contrasting agent, FIGS. 3a to 3h are images taken from day 0 to day 25. The images revealed that obstruction of the blood vessels by embolization caused the contrasting agent to non-uniformly distribute in the blood vessels and thus the occurrence of chromatic aberration.

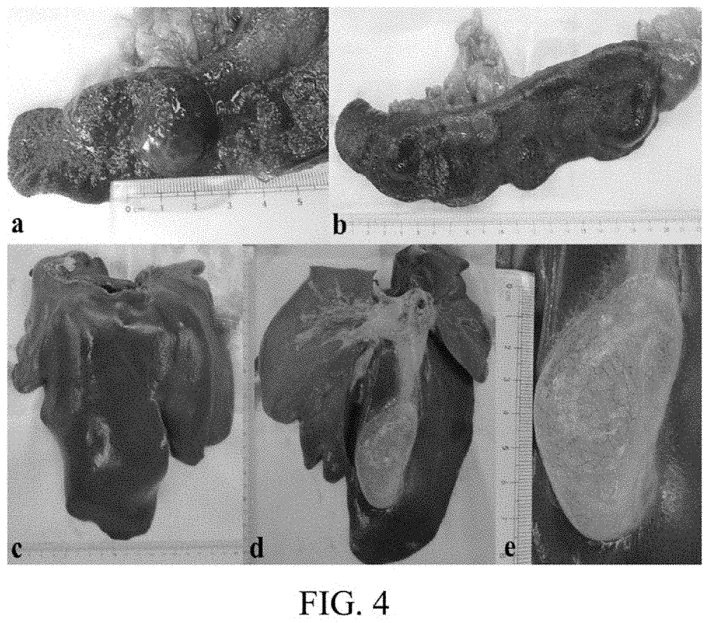

In the example of embolization using the drug-loaded microspheres as shown in FIG. 4, the pig was sacrificed after 35 days, and liver and spleen of the pig were removed. As shown in FIGS. 4a and 4b, significant atrophy of the spleen was observed. In the example, as left lobe of the liver was embolized, significant atrophy of the left and right lobes of the liver (FIGS. 4c and 4d) and enlargement of the gallbladder (FIG. 4e) were shown.

The aforementioned embodiments merely describe the principles and effects of the present invention, but are not intended to limit the present invention. A person skilled in the art can modify and change the aforementioned embodiments without departing from the spirit of the present invention. The scopes of the present invention shall be defined by the claims.

What is claimed is:

1. A microsphere, comprising:
a cross-linked hydrophilic substrate, comprising cross-linked sodium alginate and gelatin;
a lipophilic substrate, comprising iodized oil, C16-C18 alkyl alcohol, and polycaprolactone; and
a surfactant, comprising polyoxyethylene stearate,
wherein the microsphere is dry or substantially solid.

2. The microsphere according to claim 1, wherein the dry weight of the cross-linked hydrophilic, substrate is 50 to 70 wt %, the dry weight of the lipophilic substrate is 18 to 30 wt %, and the dry weight of the surfactant is 4 to 8 wt %, when the total dry weight of the microsphere is 100 wt %.

3. The microsphere according to claim 1, wherein the cross-linked hydrophilic substrate further comprises a calcium ionic cross-linking agent.

4. The microsphere according to claim 1, further comprising a supplemental component selected from a group consisting, of a cosolvent, an antioxidant, an antibacterial agent, and a stabilizer, wherein the cosolvent is selected from a group consisting of sucrose, sorbitol and glycerol; the antioxidant is sodium thiosulfate and/or 2,6-di-tert-butyl-p-cresol; the antibacterial agent is propyl p-hydroxybenzoate; and the stabilizer is cholesterol and/or sodium acetate.

5. The microsphere according to claim 4, wherein the dry weight of the cosolvent is 1 to 15 wt %, the dry weight of the antioxidant is 0.02 to 0.2 wt %, the dry weight of the antimicrobial agent is 0.1 to 0.5 wt %, and the dry weight of the stabilizer is 0.2 to 4.5 wt %, when the total dry weight of the microsphere is 100 wt %.

6. The microsphere according to claim 1, further comprising a drug-containing mixture, wherein the drug in the mixture binds with the lipophilic substrate of the microsphere.

7. The microsphere according to claim 6, wherein the drug comprises doxorubicin.

8. The microsphere according to claim 1, having an average particle size of 40 to 1000 μm.

9. A method for preparing a microsphere, the method comprising:
mixing a cross-linked hydrophilic substrate, a lipophilic substrate and a surfactant to obtain an emulsion, wherein the cross-linked hydrophilic substrate comprises cross-linked sodium alginate and gelatin; the lipophilic substrate comprises iodized oil, C16-C18 alkyl alcohol, and polycaprolactone; and the surfactant comprises polyoxyethylene stearate; and
granulating the emulsion to obtain the microsphere.

10. The method according to claim 9, wherein the dry weight of the cross-linked hydrophilic substrate is 50 to 70 wt %, the dry weight of the lipophilic substrate is 18 to 30 wt %, and the dry weight of the surfactant is 4 to 8 wt %, when the total dry weight of the microsphere is 100 wt %.

11. The method according to claim 9, wherein the cross-linked hydrophilic substrate further comprises a calcium ionic cross-linking agent.

12. The method according to claim 9, further comprising: mixing a supplemental component with the cross-linked hydrophilic substrate, the lipophilic substrate and the surfactant to obtain the emulsion,
wherein the supplemental component is selected from a group consisting of a cosolvent, an antioxidant, an antibacterial agent, and a stabilizer, wherein the cosolvent is selected from a group consisting of sucrose, sorbitol and glycerol; the antioxidant is sodium thiosulfate and/or 2,6-di-tert-butyl-p-cresol; the antibacterial agent is propyl p-hydroxybenzoate; and the stabilizer is cholesterol and/or sodium acetate.

13. The method according to claim 12, wherein the dry weight of the cosolvent is 1 to 15 wt %, the dry weight of the antioxidant is 0.02 to 0.2 wt %, the dry weight of the antimicrobial agent is 0.1 to 0.5 wt %, and the dry weight of the stabilizer is 0.2 to 4.5 wt %, when the total dry weight of the microsphere is 100 wt %.

14. The method according to claim 9, further comprising: drying the microsphere to obtain a dry microsphere.

15. The method according to claim 14, further comprising: immersing the dry microsphere in a mixture to allow the dry microsphere to absorb the mixture and expand.

16. The method according to claim 15, wherein the mixture comprises doxorubicin.

17. The microsphere according to claim 16, wherein a weight ratio of the doxorubicin to the dry microsphere is at least 1:2.5.

18. A method for embolizing tumor in a subject, comprising:
immersing a dry microsphere in a mixture to allow the dry microsphere to absorb the mixture and expand; and
administering a therapeutically effective amount of the microsphere to the tumor,
wherein the dry microsphere comprises a cross-linked hydrophilic substrate, a lipophilic substrate, and a surfactant; the cross-linked hydrophilic substrate comprises cross-linked sodium alginate and gelatin; the lipophilic substrate comprises iodized oil, C16-C18 alkyl alcohol, and polycaprolactone; and the surfactant comprises polyoxyethylene stearate.

19. The method according to claim 18, wherein the mixture comprises doxorubicin.

20. The method according to 18, wherein the tumor is a hypervascular tumor or a hyperproliferative tumor.
